(19) United States
(12) Patent Application Publication
(10) Pub. No.: US 2010/0274236 A1
(43) Pub. Date: Oct. 28, 2010

(54) APPARATUSES AND METHODS FOR APPLYING A CRYOGENIC EFFECT TO TISSUE AND CUTTING TISSUE

(76) Inventor: William S. Krimsky, Bel Air, MD (US)

Correspondence Address: Womble Carlyle Sandridge & Rice PLLC, Seventh Floor, 1401 Eye Street, N.W., Washington, DC, 20005, US

(21) Appl. No.: 12/428,925

(22) Filed: Apr. 23, 2009

(51) Int. Cl. A61B 18/02

(52) U.S. Cl. 606/21

(57) ABSTRACT

The disclosure provides a surgical device adapted to administer cryogen to a target tissue. The device may be equipped with one or more blades and the device may be adapted to deliver cryogen to a tissue before, during and/or after contacting the tissue with the blade. According to some embodiments, the device may be adapted to heat and/or cool the blade(s).

### BACKGROUND OF THE INVENTION

The application of cryogen has been used in medicine to freeze healthy or diseased tissue. Typically, a lesion or other undesired tissue is frozen using either the direct application of a cryogen or by contacting the tissue with a cryogenic probe. After freezing, the treated tissue will generally become necrotic and will slough off allowing the growth of new tissue to replace the treated tissue.

Cryotherapy has been used in combination with other surgical techniques. An example of such a combination is provided by the cryo-scalpel disclosed in U.S. Pat. No. 3,662,755. In this device, a refrigerant is injected into the blade of the scalpel in order to reduce the temperature of the scalpel. The scalpel is also equipped with a heater that permits heating of the blade to permit detachment from the frozen tissue. Since cooling of the target tissue is effected by contacting the tissue with the cooled blade, this limits the speed at which tissue is cooled. In addition, since the blade is at a temperature below the freezing point of water, the blade will tend to freeze to the tissue being cut.

There remains a need in the art for apparatuses and methods for cooling tissue that will be cut that overcomes the difficulties of the prior art. This need and others is met by the apparatuses and methods disclosed herein.

### SUMMARY OF THE INVENTION

The disclosure relates to surgical devices adapted to administer cryogen to a target tissue and cut tissue, and methods for using such a devices. The surgical devices may comprise a blade attached to a handle; wherein the handle and/or the blade include one or more cryogen delivery orifices. Typically, the orifice(s) is adapted to spray cryogen to a tissue in proximity to the blade or to create an isotherm in proximity to the blade. Any suitable cryogen may be used in conjunction with the surgical devices disclosed herein. An example of a suitable cryogen is a low temperature gas, for example, a liquefied gas. Examples of suitable cryogens include, but are not limited to, oxygen, nitrogen, argon, carbon dioxide, and air.

In some embodiments, a surgical device may be equipped with one or more blades wherein at least one blade is removably attached to the handle. According to some embodiments, the handle includes one or more cryogen delivery orifices. In some surgical devices of the invention, the blade includes one or more cryogen delivery orifices. According to further embodiments, the blade and the handle each define one or more cryogen delivery orifices.

In addition to being adapted to administer cryogen, a surgical device may also be adapted to administer heat to the device itself and/or to a target tissue which may be the same or different tissue as the tissue to which cryogen is administered. In some embodiments, a surgical device may include one or more heating elements. The heating element(s) may be adapted to heat any portion of the device. According to some the heating element(s) may be adapted to heat the blade. According to some embodiments, the heating element(s) may be adapted to heat the handle. According to further embodiments, the heating element(s) may be adapted to heat both the blade and the handle.

According to some embodiments, a surgical device may comprise one or more cooling elements. The cooling element(s) may be disposed at any position in the surgical device. In some embodiments, the cooling element(s) is adapted to use Joule-Thompson cooling. In some embodiments, the cooling element(s) may be adapted to cool the blade of the surgical device. In other embodiments, the cooling element(s) may be adapted to cool the handle. According to further embodiments, the cooling element(s) may be adapted to cool both the blade and the handle.

According to some embodiments, a surgical device may comprise one or more heating elements and one or more cooling elements.

According to some embodiments, a surgical device may comprise a cryogen supply tube attached to the device. The cryogen supply tube may be attached to any portion of the device, for example, the handle and/or the blade. Typically, the cryogen delivery orifice(s) is adapted to receive cryogen from the cryogen supply tube, and spray cryogen to a tissue or create an isotherm in proximity to the tissue. The tissue may be in proximity to the blade. Any cryogen known to those skilled in the art may be used in conjunction with a surgical device including a cryogen supply tube. An example of a suitable cryogen is a low temperature gas, for example, a liquefied gas. Examples of suitable cryogens include, but are not limited to, oxygen, nitrogen, argon, carbon dioxide, and air.

Methods of performing surgery on a patient in need thereof are also disclosed herein. According to some embodiments, a method of performing surgery may include: spraying cryogen on a tissue of the patient or creating an isotherm in proximity to the tissue with cryogen using a surgical device as described herein; and contacting the tissue with the blade. The tissue to be contacted with the blade may be treated with cryogen at any time, for example, before being contacted with the blade, while being contacted with the blade, and/or after being contacted with the blade.

According to further embodiments, a method of performing surgery may include additional heating and/or cooling of the tissue to be treated. For example, in some methods of performing surgery, a tissue may be treated by spraying cryogen on the tissue of the patient or creating an isotherm in proximity to the tissue with cryogen, and the tissue may then be heated prior to being contacted with the blade, while being contacted with the blade, and/or after being contacted with the blade. According to other embodiments, a tissue may be treated by spraying cryogen on the tissue of the patient or creating an isotherm in proximity to the tissue with cryogen and the tissue may then be further cooled prior to being contacted with the blade, while being contacted with the blade, and/or after being contacted with the blade.

The methods of performing surgery may be used to treat any type of tissue. Examples of tissues to be treated using the methods of the invention include, but are not limited to, epithelial tissue, connective tissue, muscle tissue and/or nervous tissue.

Other features and advantages will become apparent to those skilled in the art from the following description and appended drawings.

### BRIEF DESCRIPTION OF THE DRAWINGS

FIG. 1A is a side view of a surgical device according to an embodiment.

FIG. 1B is a sectional view of the surgical device of FIG. 1A.

FIG. 2A is a sectional view of a surgical device according to another embodiment.

FIG. 2B is a rear end view showing a handle of the surgical device of FIG. 2A.

FIG. 3 is a sectional view of a surgical device according to another embodiment.

FIG. 4 is a perspective view of a surgical device according to another embodiment.

FIG. 5 is sectional view of a handle for a surgical device according to another embodiment.

FIG. 6 shows another embodiment, in which a surgical device includes a blade adapted to be heated and/or cooled during use.

FIG. 7A is a partial side view of a surgical device according to another embodiment.

FIG. 7B is an end view showing a blade of the device of FIG. 7A.

### DETAILED DESCRIPTION OF THE INVENTION

The following description discloses various embodiments of surgical devices for applying cryogenic material (“cryogen”) to tissue and cutting tissue. In the various embodiments that follow, reference characters shared among the embodiments indicate similar components and/or features. The surgical devices disclosed herein may be used in conjunction with any known system, method and/or device for the delivery of cryogen to a target tissue. Examples of suitable devices for the delivery of cryogen include, but are not limited to, those disclosed in U.S. Pat. Nos. 6,027,499, 6,383,181, 7,025,762, and 7,255,693 issued to Johnston et al., the disclosures of which are specifically incorporated herein by reference for their disclosures of a cryogen delivery device.

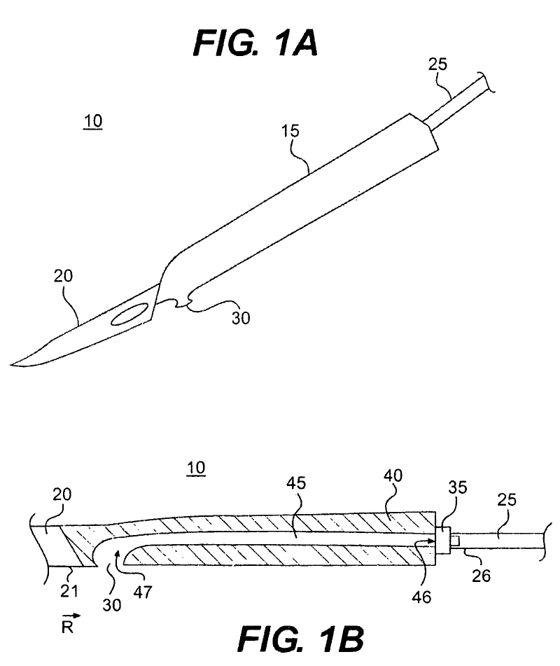

With reference to FIG. 1A, a surgical device 10 is depicted. The surgical device 10 is includes a handle 15 and a blade 20. A cryogen supply tube 25 is attached to the handle 15 and is in fluid communication with a cryogen storage device, for example, a container of liquefied gas (not shown). The handle 15 includes a cryogen delivery orifice 30 for spraying a cryogen delivered by the cryogen supply tube 25. Referring to FIG. 1B, the surgical device 10 may include a fluid channel or cryogen channel 45 for flowing a cryogen received from the cryogen supply tube 25. The channel 45 may have a first end 46 for receiving the cryogen from the delivery tube 25 and a second end 47 terminating at the cryogen delivery orifice 30. The channel 45 may be an elongate, open pathway, and may have any suitable cross-sectional shape. It should be understood that, although a single cryogen channel 45 and a single orifice 30 are shown, it is possible for the handle 15 to include multiple orifices 30 or multiple cryogen channels 45 each terminating at one or more orifices 30.

The exit end 26 of the cryogen supply tube 25 is shown attached to the handle 15 via a fitting 35. The fitting 35 may be any fitting suitable for attaching the cryogen supply tube 25 to the handle 15. Examples of suitable fittings include, but are not limited to, a luer fitting, a threaded fitting, a compression fitting and the like. However, it should be understood that other means for attaching the cryogen supply tube 25 to the handle 15 are possible. For example, a fitting may be provided inside of the channel 45 for attaching the tube 25, or the exit end 26 of the tube 25 may be inserted directly into the channel 45 and retained in the channel 45 by a friction fit or an adhesive bond with the walls of the channel 45.

As shown in FIG. 1B, the internal portion of handle 15 may include an insulating material 40 surrounding the cryogen channel 45. The insulating material 40 may be the same material that is used to form remaining portions of the handle 15, or it may be a different material. Any material that is suitable to insulate the user's hand from the cryogen delivered through the handle may be used. Suitable examples of insulating materials that may be used include, but are not limited to, polycarbonate, fluoropolymer, polyimide, polyetheretherketone (PEEK), polyetherimide/ULTEM, polyamide-imide, and combinations thereof. In some embodiments, combinations of materials may be used to fabricate the handle. In some embodiments, different materials may be used to fabricate different portions of the handle, for example, portions of the handle that will be exposed to low temperatures (e.g., portions close to the blade and or cryogen orifices) may be fabricated with one material and another material may be used to fabricate the portion of the handle contacted by the user, for example, a non-slide material may be used to fabricate all or a portion of the handle gripped by the user.

In operation of the device 10, cryogen flows from a cryogen storage device (not shown) through the cryogen supply tube 25, then is delivered to the cryogen channel 45 from the cryogen supply tube 25 via the fitting 35, then flows through the cryogen channel 45, and then exits the handle 15 through the cryogen delivery orifice 30.

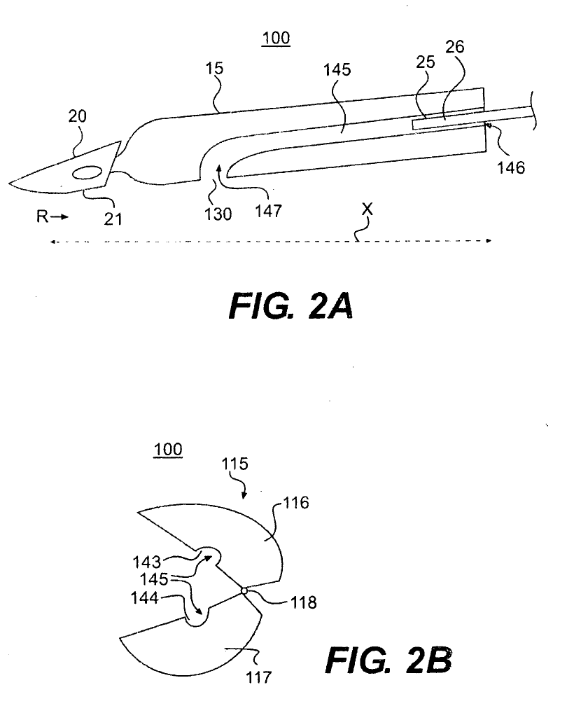

FIGS. 2A and 2B show a surgical tool 100 according to another embodiment.

As shown in FIG. 2A, the tool 100 includes a handle 115 and a blade 20 attached to the handle 115. The handle 115 defines an interior cryogen channel 145 that is generally similar to the channel 45 described in the previous embodiment. The cryogen channel 145 has a first end 146 for receiving an exit end 26 of a cryogen supply tube 25 and a second end 147 that terminates at a cryogen delivery orifice 130. Referring to FIG. 2B, which shows the handle 115 in an open position for receiving the cryogen supply tube 25, the handle 115 is formed by two mating handle portions 116, 117 that are at least partially separable along the axial direction X of the handle generally corresponding to the axial path of the cryogen channel 145. As illustrated in FIG. 2B, the portions 116, 117 may be pivotally attached to each other by a longitudinally extending hinge or pivot connection 118. The pivot connection 118 may be formed of any material and may be integral to the handle 115. Alternatively, the pivot connection 118 may be a separate part and the handle 115 may be formed of the two portions 116, 117 with the pivot connection 118 attached to both of the portions 116, 117. In some embodiments, the pivot connection 118 may be formed of the material used to form the remainder of the handle 115. For example, the pivot connection 118 may be a flexible portion of the material of the handle 115 that allows the handle 115 to be opened and closed. The portions 116, 117 may include respective channel segments 143, 144 that combine to form the cryogen channel 145 when the handle 115 is closed.

To attach the cryogen supply tube 25 to the handle 115, the handle 115 can be opened by pivoting the portions 116, 117 apart about the pivot connection 118, the cryogen supply tube 25 can then be aligned with the channel segments 143, 144, and the handle 115 can then be closed around the cryogen supply tube 25 by pivoting the portions 116, 117 towards each other and securing the portions 116, 117 together such that the cryogen supply tube 25 is positioned in the cryogen channel 145. The portions 116, 117 may be secured together by any suitable mechanism such as a detent mechanism with interlocking male and female latching or locking members (not shown). The cryogen supply tube 25 may be conveniently removed from the handle 115 if desired by opening the handle 115. In this embodiment, the exit end 26 of the cryogen supply tube 25 is retained in the cryogen channel 145 by a friction fit with the walls of the channel 145. However, according to other embodiments, the cryogen supply tube 25 may be removably or permanently affixed in position, for example, by attaching the cryogen supply tube 25 to a fitting (not shown) or adhesive placed in the cryogen channel 145. According to additional embodiments, the pivot connection 118 may be eliminated such that the handle segments 116, 117 are completely separable from each other and can be secured together by simply pressing the segments 116, 117 together to engage one or more detent mechanisms.

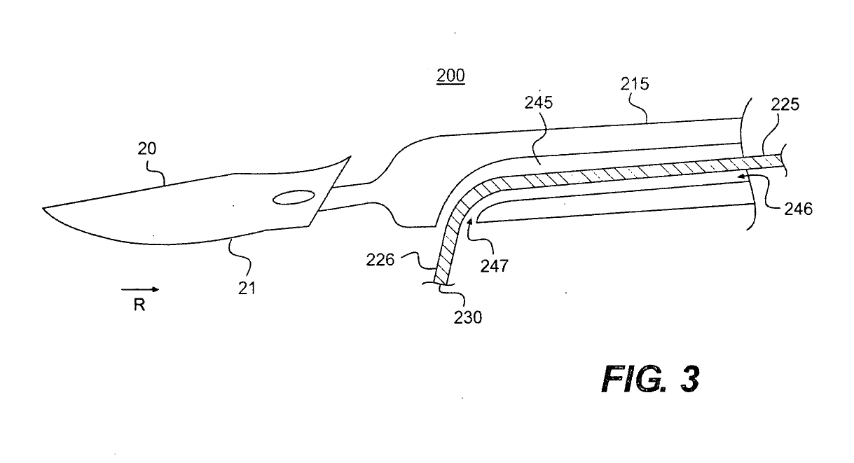

FIGS. 1A-1B and FIGS. 2A-2B depict embodiments in which the cryogen delivery orifice is formed as part of handle. However, other arrangements are envisioned. For example, a cryogen delivery orifice may be a separate part attached to the end of a cryogen supply tube or may be an opening in cryogen supply tube, as shown in FIG. 3. Referring to FIG. 3, a surgical device 200 according to another embodiment includes a handle 215 and a blade 20. The surgical device 200 includes an elongate open channel 245 formed in an interior region of the handle 215 and a cryogen supply tube 225 extending through the channel 245. Thus, the channel 245 retains and guides the cryogen supply tube 225. The channel 245 includes a first end 246 of that defines an entry way through which the tube 225 can be inserted into the channel 245, and a second end 247 that defines and exit through which an exit end 226 of the tube 225 protrudes from the handle 215. The cryogen supply tube 225 may include one or more cryogen delivery orifices 230. The cryogen delivery orifice 230 may be formed by an opening in the cryogen supply tube 225, or it may be formed by a fitting or nozzle attached to the exit end 226 of the tube 225. It should be understood that, although only one channel 245 and associated cryogen supply tube 225 are shown, it is possible to provide the handle 215 with multiple channels 245 each having a cryogen supply tube 225 therein, or a single channel 245 having multiple cryogen supply tubes 225 therein. It should also be understood that the handle 215 may optionally be formed by at least partially separable segments in a manner similar to that described with respect to the embodiment of FIGS. 2A and 2B.

In operation of the device 200, cryogen is delivered through the cryogen supply tube 225, and then exits the tube 225 through the cryogen delivery orifice 130.

In each of the embodiments depicted in FIGS. 1A-3, the cryogen delivery orifice 30/130/230 is positioned at an underside of the handle 15/115/215 in order to apply cryogen to tissue below the handle, typically in the cutting path or vicinity of the cutting edge 21 of the blade 20, which is located at a bottom surface of the blade 20. It should be understood however, that the orifices 30, 130, 230 may be positioned at any desired location in or on the respective handles 15, 115, 215. It should also be understood that, although the cryogen channels 45, 145, 245 are shown and described as being located in the interior space of the handles 15, 115, 215, the cryogen channels 45, 145, 245 may be formed as tubes attached to or formed on an outer wall of the handles 15, 115, 215.

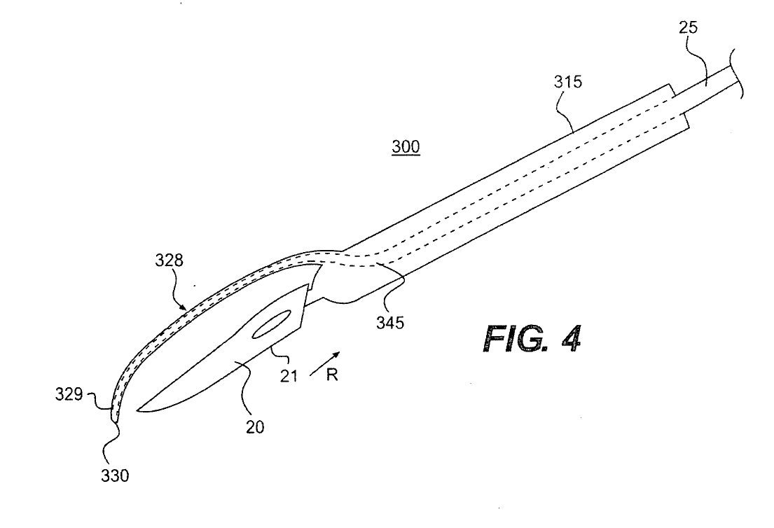

In the embodiments shown in FIGS. 1A-3, the surgical devices 10, 100, 200 are configured to facilitate delivering cryogen to a target tissue prior to contacting the tissue with blade 20, due to the fact that the blade 20 is positioned in front of the cryogen delivery orifice 30/130/230 and the blade is typically used in a rearward cutting motion indicated by the direction R (FIGS. 1B, 2A and 3). One skilled in the art will appreciate that it may also be desirable to contact tissue during and/or after contacting the tissue with the blade. FIG. 4, which is described below, shows as surgical device 300 configured to facilitate delivering cryogen to a target tissue after the tissue has been contacted with the blade 20. Although the devices 10, 100, 200 are stated to be configured for delivering cryogen to tissue before cutting the tissue and the device 300 is stated to be configured for delivering cryogen to tissue after cutting the tissue, it should be understood that each of the devices 10, 100, 200, 300 can be used for delivering cryogen to tissue before, during or after cutting the tissue.

Referring to FIG. 4, the surgical device 300 includes a handle 315 and a blade 20 attached to the handle 315. A cryogen supply tube 25 is attached to the handle 315.

The tube 25 may be directly attached to the handle 315 by a friction fit or adhesive bond, or by an internal or external fitting as described with respect to previous embodiments.

The device 300 also includes a cryogen application tube 328 attached to and extending from the handle 315 and extending forward from the handle 315 and over top of the blade 20. The cryogen application tube 328 may be formed integrally with the handle 315, or alternatively, may be attached to the handle 315 by a fitting (not shown), an adhesive bond, or a friction fit within an opening (not shown) in the handle 315. The cryogen application tube 328 includes an exit end 329 and a cryogen delivery orifice 330 at the exit end 329. A cryogen channel 345 extends through the interior of the handle 315 and the cryogen application tube 328. Alternatively, the cryogen channel 345 may be formed as tube attached to or formed on an outer wall of the handle 315.

The channel 345 is in fluid communication with the cryogen supply tube 25 and terminates at the orifice 330. In operation of the device 300, cryogen is delivered through the cryogen supply tube 25, and then exits the tube 25 through the cryogen delivery orifice 330.

As shown in FIG. 4, the exit end 329 of the cryogen application tube 328 may be positioned in front of the forward tip of the blade 20, such that cryogen can be applied to tissue through the orifice 330 in proximity to the blade 20 after the tissue has been cut using a rearward (R) cutting motion. In this embodiment, the cryogen application tube 328 extends over top of the blade 20 so as not to interfere with the cutting edge 21 of the blade 20, which is located at the bottom of the blade 20. One skilled in the art will appreciate that the cryogen application tube 328 can be configured and/or positioned so as to spray cryogen onto the blade for example, in the middle of the blade 20. The cryogen application tube 328 may optionally be formed from a deformable material, including but not limited to a flexible rubber, plastic, metal or steel material, such that the exit end 329 of the cryogen application tube 328 can be selectively placed in various positions with respect to the blade 20. It should also be understood that the cryogen application tube 328 can be incorporated in other devices, such as the devices shown in FIGS. 1-3.

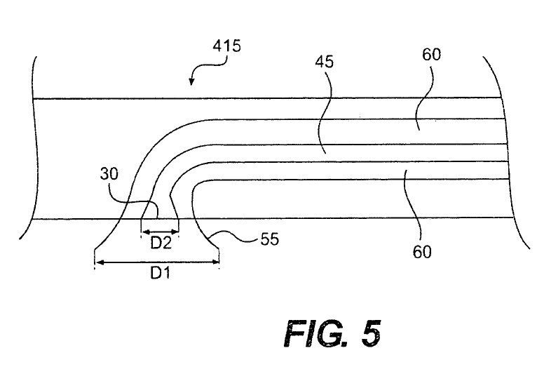

In FIG. 5, a portion of a handle 415 for a surgical device similar to the devices of the previous embodiments is shown. The handle 415 is equipped with a suction hood 55. In the embodiment shown, the suction hood 55 is disposed around a cryogen delivery orifice 30. One skilled in the art will appreciate that suction hood 55 may be placed in other relationships to the cryogen delivery orifice 30. For example, the cryogen delivery orifice 30 may be adjacent to the suction hood 55. The diameter D1 of the suction hood 55 may be of any size, i.e., it may be larger, the same size, or smaller than the diameter D2 of the cryogen delivery orifice 30. A surgical device may be equipped with a plurality of suction hoods that may be the same or different sizes. Typically, when a handle such as the handle is provided with a suction hood, the handle will also be provided with a suction tube or channel for applying suction in proximity to target tissue. In the embodiment shown in FIG. 5, a suction tube, passage or channel 60 is shown disposed around the cryogen channel 45. The suction channel 60 is in fluid communication with a suction pump or another conventional device (not shown) for creating suction. One skilled in the art will appreciate that the suction channel 60 may have any spatial relationship with regard to the cryogen channel 45, e.g., may run along side of the channel 45 or may run along the outside of the handle 415. In one embodiment, the suction channel 60 may be formed in the handle 415 and is coaxial with the channel 45.

In operation, cryogen may be delivered through the channel 45 and may exit the cryogen delivery orifice 30. At the same time, suction may be applied to the suction channel 60. The handle 415 may be oriented such that the suction hood 55 is in proximity to, but not in contact with, the target tissue. As the cryogen exits the cryogen delivery orifice 30, it may contact the target tissue or create an isotherm in proximity to the target tissue and then be drawn up into the suction hood 55 which then directs the cryogen into the suction channel 60. One skilled in the art will understand that a suction hood and suction channel such as the suction hood 55 and suction channel 60 in the instant embodiment may employed in any of the other embodiments described herein.

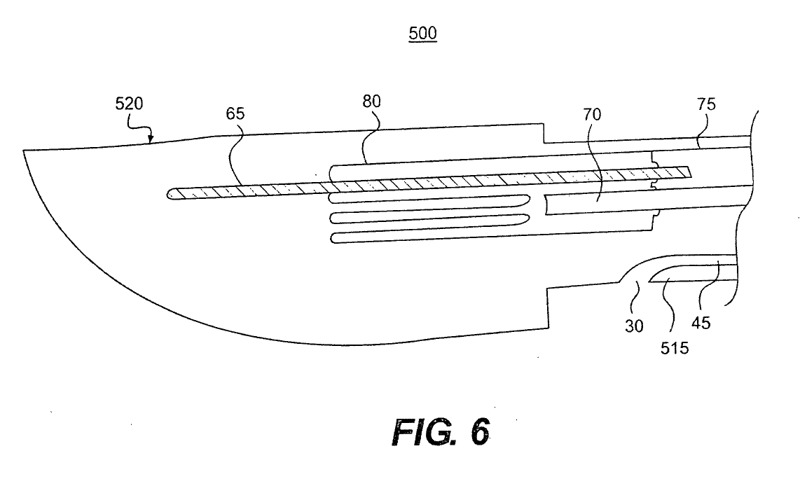

In some embodiments, a blade of a surgical device may be a heated and/or cooled blade. FIG. 6 shows a partial view of a surgical device 500 including a blade 520 that can be heated and cooled, and an handle 515 attached to the blade 520. The handle 515 includes a cryogen channel 45 and a cryogen delivery orifice 30. The blade 520 includes an auxiliary cryogen supply tube 65 that is configured to deliver cryogen to an interior space of the blade 520. The auxiliary cryogen supply tube 65 may be in fluid communication with the same source of cryogen used by another, primary cryogen supply tube such as the cryogen supply tubes 25, 225, described in previous embodiments, or it may be in fluid communication with a different cryogen source. The auxiliary cryogen supply tube 65 is adapted to deliver cryogen in the form of liquefied gas. Those skilled in the art will appreciate that auxiliary cryogen supply tube 65 could be equipped with a nozzle (not shown) with a suitable aperture and cooling could be effected by forcing pressurized gas through the nozzle and utilizing the Joule-Thomson effect.

The blade 520 is also equipped with a suction tube or channel 70. The auxiliary cryogen supply tube 65 is in fluid communication with a cryogen source, the interior space of blade 520, and the suction channel 70. The suction channel 70 may extend through the handle 515 and may be connected to a suction device (not shown). In use, cryogen may be delivered to the interior space of the blade 520 to effect cooling of the blade and then may exit the interior space of the blade 520 via the suction tube.

The device 500 also includes an electrical connection 75 connected to a heating element 80 disposed in the blade 520. Electrical current can be caused to flow through the heating element 80, resulting in heating of the blade 520. The blade 520 may be heated and/or cooled as necessary or desirable during the course of surgery by causing electricity to flow through the heating element and/or cryogen to flow through an auxiliary cryogen supply tube 65. It should be understood that the blade 520 could be modified, if desired, to include only heating components or cooling components, as opposed to both heating and cooling components as shown in FIG. 6.

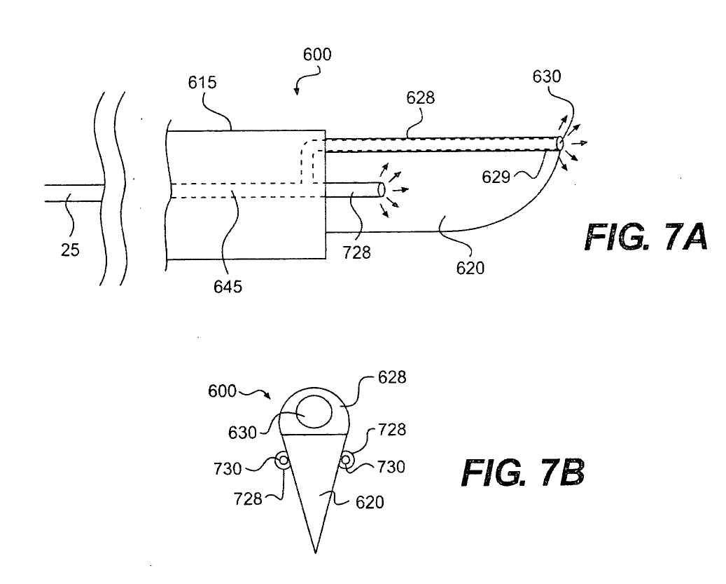

FIGS. 7A and 7B show surgical device 600 according to another embodiment. The device 600 is particularly configured for spraying cryogen on or in proximity to tissue while the tissue is being cut, but may also be used to spray cryogen on tissue before or after cutting the tissue. The surgical device 600 includes a handle 615 and a blade 620 attached to the handle 615. The blade 620 may include a cryogen application tube 628 generally extending along a top edge of the blade 620, and a pair of cryogen application tubes 728 generally extending along the side surfaces of the blade 620. The cryogen application tubes 628, 728 include respective cryogen delivery orifices 630, 730 at exit ends 629, 729 of the tubes 628, 728. The cryogen application tubes 628, 728 may be separate parts attached to the blade 620, or they may be integrally formed with the blade 620. The orifices 630, 730 may be in fluid communication with a cryogen channel 645 extending through or along the handle 615. The cryogen channel 645 may be connected to a cryogen supply tube 25 as described with respect to preceding embodiments discussed herein. Alternatively, the cryogen channel 645 may be eliminated and one or more cryogen supply tubes similar to the illustrated tube 25 may extend within or along the handle 615 and be connected directly to the cryogen application tubes 628, 728. The cryogen application tubes 628, 728 can both spray target tissue and cool the blade 620 due to their positioning. It is also possible to provide a blade having either the tube 628 or tubes 728, rather than both the tube 628 and tubes 728. One skilled in the art will also understand the blade 620 may also be used with the handles 15, 115, 215, 315, 415 disclosed in other embodiments herein.

Each of the embodiments disclosed herein is shown as having a static knife-type blade 20/520/620. However, it is possible to provide other types of blades. For example, a motorized reciprocating blade, a serrated blade, or a rotary blade may be provided.

The surgical devices 100, 200, 300, 500, 600 described above may be connected to a cryogen storage device, such as a tank of liquefied gas, for supplying cryogen to the surgical devices. The cryogen storage device may be a component of a cryogen delivery system capable of controlling the flow of cryogen to the cryogen supply tube 25/225. Typically, the flow of cryogen from the cryogen storage device to the surgical device 100/200/300/500/600 may be controlled using any suitable valve known to those skilled in the art. Such a valve is typically equipped with an actuator and is capable of being actuated remotely by a control device, for example, by a foot pedal and/or hand operated button. In operation, the valve is actuated to open the flow path from the cryogen storage device through the cryogen supply tube 25/225, thereby causing cryogen to travel from the cryogen storage device through the cryogen supply tube 25/225 and exit out of the cryogen delivery orifice 30/130/230/330.

The devices 100/200/300/500/600 disclosed herein may be used, for example, to conduct surgery by spraying cryogen on a tissue of a patient or creating an isotherm in proximity to the tissue with cryogen and contacting the tissue with the blade 20/520/620. The tissue to be contacted with the blade 20/520/620 may be treated with cryogen at any time, for example, before being contacted with the blade, while being contacted with the blade, and/or after being contacted with the blade. When the device 500 is employed, surgery may be performed by spraying cryogen on the tissue of the patient or creating an isotherm in proximity to the tissue with cryogen, and heating the tissue prior to contacting the tissue with the blade 520, while contacting the tissue with the blade 520, and/or after contacting the tissue with the blade 520. When the devices 500, 600 are employed, a tissue may be treated by spraying cryogen on the tissue or creating an isotherm in proximity to the tissue with cryogen, and further cooling the tissue prior to contacting the tissue with the blade, while contacting the tissue with the blade, and/or after contacting the tissue with the blade.

While the disclosure specifically discusses devices and methods for applying cryogen to tissue, the disclosure is not limited to the application of cryogen treatments. Fluids other than cryogen may be employed in the disclosed devices and methods for treating tissue that is to be cut.

While the foregoing invention has been described in specific exemplary embodiments, it will be appreciated by one skilled in the art from a reading of this disclosure that various changes in form and detail can be made without departing from the true scope of the invention and appended claims. All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A surgical device, comprising:
a handle;
a blade attached to the handle; and
at least one orifice configured to spray a fluid from the surgical device, the at least one orifice being disposed in or on at least one of the handle and the blade.

2. The surgical device of claim 1, wherein the at least one orifice comprises an orifice positioned at one of the following locations: an underside of the handle, in front of a tip of the blade, a side surface of the blade, and a top edge of the blade.

3. The surgical device of claim 1, wherein the treatment fluid is cryogen, wherein the least one orifice is in fluid communication with a source of the cryogen, and wherein the at least one orifice is configured to spray the cryogen to a tissue in proximity to the blade or to create an isotherm in proximity to the blade.

4. The surgical device of claim 3, wherein the cryogen is a liquefied gas.

5. The surgical device of claim 3, wherein the cryogen is selected from the group consisting of oxygen, nitrogen, argon, carbon dioxide, and air.

6. The surgical device of claim 3, comprising at least one channel configured to direct the cryogen to the at least one orifice, wherein the at least one channel extends through an interior of the handle or is disposed on an outer wall of the handle.

7. The surgical device of claim 6, comprising a cryogen supply tube configured to be placed in fluid communication with a source of the cryogen, wherein the at least one channel is configured to be placed in fluid communication with the cryogen supply tube.

8. The surgical device of claim 6, wherein the at least one orifice is disposed in a cryogen application tube attached to or disposed on the handle or the blade, and wherein the at least one channel comprises a channel extending through the cryogen application tube.

9. The surgical device of claim 6, comprising a suction passage configured to suction an excess portion of the cryogen after the cryogen is sprayed.

10. The surgical device of claim 9, wherein the suction passage is coaxial with or adjacent to the channel.

11. The surgical device of claim 6 comprising a suction hood configured to direct the excess portion of the cryogen into the suction channel.

12. The surgical device of claim 11, wherein the suction hood is disposed around the at least one orifice.

13. The surgical device of claim 1, comprising at least one channel configured to direct the treatment fluid to the at least one orifice, wherein the at least one channel extends through an interior of the handle or is disposed on an outer wall of the handle.

14. The surgical device of claim 13, comprising a fluid supply tube configured to be placed in fluid communication with a source of the treatment fluid, wherein the at least one channel is configured to be placed in fluid communication with the fluid supply tube.

15. The surgical device of claim 13, wherein the at least one orifice is disposed in a fluid application tube attached to or disposed on the handle or the blade, and wherein the at least one channel comprises a channel extending through the fluid application tube.

16. The surgical device of claim 6, wherein the at least one orifice is disposed in a liquid application tube attached to or disposed on the handle or the blade, and wherein the at least one channel comprises a channel extending through the liquid application tube.

17. The surgical device of claim 1, comprising at least one heating element configured to heat the blade.

18. The surgical device of claim 1, wherein the blade comprises at least one cooling element.

19. The surgical device of claim 14, wherein the at least one cooling element comprises at least one tube adapted to carry a cooling fluid.

20. The surgical device of claim 15, wherein the cooling fluid is the treatment fluid.

21. The surgical device of claim 1, wherein the blade is removably attached to the handle.

22. The surgical device of claim 1, wherein the fluid is cryogen, an wherein the device comprises a suction passage configured to suction an excess portion of the cryogen after the cryogen is sprayed.

23. The surgical device of claim 22, comprising a suction hood configured to direct the excess portion of the cryogen to the suction passage.

24. The surgical device of claim 23, wherein the suction hood is disposed around the at least one orifice.

25. A surgical device, comprising:
a handle;
a blade attached to the handle;
a fluid supply tube attachable to the handle; and
at least one orifice configured to spray a treatment fluid delivered through the fluid supply tube.

26. The surgical device of claim 25, wherein the at least one orifice is positioned at one or more of the following locations: an underside of the handle, in front of a tip of the blade, a side surface of the blade, and a top edge of the blade.

27. The surgical device of claim 25, comprising a channel extending through the handle or disposed on the handle, wherein the channel is configured to retain the fluid supply tube.

28. The surgical device of claim 25, wherein the fluid supply tube is configured to be placed in fluid communication with the channel, and wherein the at least one orifice is disposed at an end of the channel.

29. The surgical device of claim 25, comprising a channel extending through the handle or disposed on the handle, wherein the fluid supply tube is configured to extend through the channel such that a dispensing end of the fluid supply tube protrudes from the handle through an end of the channel, and wherein the at least one orifice comprises an orifice disposed at the dispensing end of the fluid supply tube.

30. The surgical device of claim 25, wherein the treatment fluid is cryogen, wherein the fluid supply tube is configured to be placed in fluid communication with a source of the cryogen, and wherein the at least one orifice is configured to spray the cryogen to a tissue in proximity to the blade or to create an isotherm in proximity to the blade.

31. The surgical device of claim 30, wherein the cryogen is a liquefied gas.

32. The surgical device of claim 31, wherein the cryogen is selected from the group consisting of oxygen, nitrogen, argon, carbon dioxide, and air.

33. The surgical device of claim 25, comprising at least one heating element configured to heat the blade.

34. The surgical device of claim 25, wherein the blade comprises at least one cooling element.

35. The surgical device of claim 34, wherein the at least one cooling element comprises at least one auxiliary fluid supply tube adapted to carry a cooling fluid.

36. The surgical device of claim 34, wherein the cooling fluid is the treatment fluid.

37. The surgical device of claim 25, wherein the blade is removably attached to the handle.

38. The surgical device of claim 25, wherein the handle comprises a first handle portion and a second handle portion, wherein the first and second handle portions are at least partially separable from each other along an axial direction of the handle for inserting the fluid supply tube between the first and second handle portions, and wherein the first and second handle portions can be secured together to retain the fluid supply tube in the handle between the first and second handle portions.

39. The surgical device of claim 38, wherein the handle includes an interior channel partially defined by the first handle portion and partially defined by the second handle portion, and wherein the interior channel is configured to receive the fluid supply tube when the first handle portion and the second handle portion are secured together.

40. The surgical device of claim 39, wherein the interior channel is configured to retain the fluid supply tube by a friction fit.

41. The surgical device of claim 38, wherein the first handle portion and the second handle portion are connected to each other by a pivot connection, and wherein the first handle portion and the second handle portion can be partially separated from each other and secured together by pivoting the first handle portion and the second handle portion with respect to each other.

42. A method of performing surgery on a patient in need thereof, comprising:
spraying cryogen on a tissue of the patient or creating an isotherm in proximity to the tissue using a surgical device according to claim 1; and
contacting the tissue with the blade either before, while or after spraying the cryogen on the tissue or creating an isotherm in proximity to the tissue.

43. The method of claim 42, wherein the tissue is selected from the group consisting of epithelial tissue, connective tissue, muscle tissue and nervous tissue.

44. The method of claim 42, comprising heating the tissue either before, while or after contacting the tissue with the blade.

45. The method of claim 42, further comprising cooling the tissue.

46. The method of claim 45, wherein cooling the tissue comprises contacting the tissue with the blade upon cooling the blade.

47. The method of claim 45, wherein cooling the tissue comprises cooling the tissue before, while or after contacting the tissue with the blade.

48. The method of claim 42, further comprising cooling the blade either before, while or after contacting the tissue.

49. A method of performing surgery on a patient in need thereof, comprising:
spraying cryogen on a tissue of the patient or creating an isotherm in proximity to the tissue using a surgical device according to claim 25; and
contacting the tissue with the blade either before, while or after spraying the cryogen on the tissue or creating an isotherm in proximity to the tissue.

50. The method of claim 49, wherein the tissue is selected from the group consisting of epithelial tissue, connective tissue, muscle tissue and nervous tissue.

51. The method of claim 49, comprising heating the tissue either before, while or after contacting the tissue with the blade.

52. The method of claim 49, further comprising cooling the tissue.

53. The method of claim 52, wherein cooling the tissue comprises contacting the tissue with the blade upon cooling the blade.

54. The method of claim 53, wherein cooling the tissue comprises cooling the tissue before, while or after contacting the tissue with the blade.

55. The method of claim 49, further comprising cooling the blade either before, while or after contacting the tissue.
